(19) United States
(12) Patent Application Publication
(10) Pub. No.: US 2010/0285075 A1
(43) Pub. Date: Nov. 11, 2010

(54) Novel Hemioxalate Salt of Eletriptan

(75) Inventors: Vijaya Gopal Kusumba, Andhra Pradesh (IN); Sampath Kumar Sankineni, Andhra Pradesh (IN); Praveen Kumar Neela, Andhra Pradesh (IN); Nitin Sharadchandra Pradhan, Maharashtra (IN); Jon Valgeirsson, Hafnarfjordur (IN)

Correspondence Address: CANTOR COLBURN, LLP, 20 Church Street, 22nd Floor, Hartford, CT, 06103, US

(73) Assignee: ACTAVIS GROUP PTC EHF, Hafnarfjordur (IS)

(21) Appl. No.: 12/808,762

(22) PCT Filed: Dec. 17, 2008
(86) PCT No.: PCT/IB08/03721

§ 371 Date: Jul. 22, 2010

(30) Foreign Application Priority Data
Dec. 17, 2007 (IN) 3004/CHE/2007

(51) Int. Cl. A61K 31/4045; C07D 403/06; A61P 25/24; A61P 25/22; A61P 3/00; A61P 3/04; A61P 25/30; A61P 29/00; A61K 9/14

(52) U.S. Cl. 424/400; 548/468; 514/414

(57) ABSTRACT

The present invention relates to novel hemioxalate salt of eletriptan, process for preparation, pharmaceutical compositions, and method of treating thereof. The present invention relates to solid forms of eletriptan hemioxalate, processes for preparation, pharmaceutical compositions, and method of treating thereof. The solid form of eletriptan hemioxalate is useful for preparing eletriptan free base or a pharmaceutically acceptable salt thereof, particularly eletriptan hydrobromide, in high purity. The present invention also provides a process for preparing substantially pure eletriptan hydrobromide using eletriptan hemioxalate.

### CROSS REFERENCE TO RELATED APPLICATION

This application claims the benefit of priority to Indian provisional application No. 3004/CHE/2007, filed on Dec. 17, 2007, which is incorporated herein by reference.

### FIELD OF THE DISCLOSURE

The present invention relates to novel hemioxalate salt of eletriptan, process for preparation, pharmaceutical compositions, and method of treating thereof. The present invention further relates to solid forms of eletriptan hemioxalate, processes for preparation, pharmaceutical compositions, and method of treating thereof. The solid form of eletriptan hemioxalate is useful for preparing eletriptan free base or a pharmaceutically acceptable salt thereof, particularly eletriptan hydrobromide, in high purity. The present invention also provides a process for preparing substantially pure eletriptan hydrobromide using eletriptan hemioxalate.

### BACKGROUND OF THE INVENTION

Eletriptan, chemically named (R)-5-[2-(phenylsulfonyl)ethyl]-3-[(1-methyl-2-pyrrolidinyl)methyl]-1H-indole, is a selective 5-hydroxytryptamine 1B/1D (5-HT1B/1D) receptor agonist and may be used in the treatment of depression, anxiety, eating disorders, obesity, drug abuse, cluster headache, migraine, chronic paroxysmal hemicrania and headache associated with vascular disorders, pain, and other disorders arising from deficient serotonergic neurotransmission. Eletriptan is represented by the following structural formula:

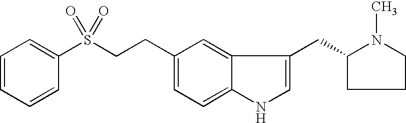

and its first synthesis was disclosed in U.S. Pat. No. 5,545,644 (hereinafter referred to as the '644 patent). Eletriptan is sold by Pfizer under the brand name RELPAX® for the treatment of migraine headaches. It is orally administered as tablets containing 24.2 mg or 48.5 mg of eletriptan hydrobromide equivalent to 20 mg or 40 mg of eletriptan.

Salts of eletriptan, including the hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate salts, have been described in the literature. Eletriptan has been exemplified as a free base and hemisuccinate salt in the '644 patent. The '644 patent also discloses four solid forms (α-form, β-form, o-form and s-form) of eletriptan hydrobromide salt, compositions comprising the solid forms, methods of making the solid forms and methods of use thereof.

While the '644 patent generally mentions that the basic compounds disclosed in that patent can form a salt with pharmaceutically acceptable organic or inorganic acids like hydrochloric acid, hydrobromic acid, hydroiodic acid, nitric acid, sulfuric acid, phosphoric acid, acetic acid, lactic acid, citric acid, tartaric acid, succinic acid, maleic acid, fumaric acid, gluconic acid, saccharic acid, benzoic acid, methanesulfonic acid and pamoic acid, only the hydrobromide and hemisuccinate salts of eletriptan have been prepared and/or isolated.

U.S. Pat. No. 6,110,940 (hereinafter referred to as the '940 patent) discloses two crystalline forms (α-form and β-form) of eletriptan hydrobromide, processes for the preparation, and characterizes them by powder X-ray diffraction (P-XRD), infra-red spectroscopy (IR) and Differential Scanning Calorimetry (DSC).

The '940 patent also teaches that attempts have been made to obtain a suitable form of the following salts: hydrochloride, hydrobromide, hemisulphate, bisulphate, nitrate, acid phosphate, phosphate, methanesulphonate, benzenesulphonate, p-toluenesulphonate, (+)-camphorsulphonate, acetate, benzoate, citrate, hemifumarate, fumarate, hemimaleate, maleate, hemisuccinate, succinate, hemi-L-tartrate, L-tartrate, hemi-D-tartrate, D-tartrate, L-lactate, (R)-(−)-mandelate, hippurate, hemiphthalate, phthalate and hemiterephthalate. Of these thirty possible salts, only four could be obtained as crystalline solids, namely the hemisulphate, hydrochloride, hydrobromide and benzenesulphonate; the remainder were obtained as non-crystalline/low or non-sharp melting/sticky solids, gums, glasses, froths, resins or oils. Moreover, of the four crystalline salts, the benzenesulphonate proved to have an insufficiently high melting point (m.p.) of 74-75° C.

European Patent No. 1135381 B1 and subsequent equivalent U.S. Pat. No. 7,238,723 B2 disclose a crystalline monohydrate of eletriptan hydrobromide and processes for its preparation.

U.S. Patent Application No. 2004/0204475 A1 (hereinafter referred to as the '475 application) is directed to a combination comprising eletriptan, or a pharmaceutically acceptable salt thereof, and sodium bicarbonate, the use of such a combination for the treatment or prevention of a disease for which a 5-HT₁ agonist is indicated and formulations and products comprising such a combination. While the '475 application mentions that eletriptan can form salts with acids, such as acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts; no salts of the eletriptan have been prepared or isolated.

In the formulation of drug compositions, it is important for the active pharmaceutical ingredient to be in a form in which it can be conveniently handled and processed. This is of critical, not only from the point of view of obtaining a commercially viable manufacturing process, but also from the point of view of subsequent manufacture of pharmaceutical formulations (e.g. oral dosage forms such as tablets) comprising the active pharmaceutical ingredient.

Further, in the manufacture of oral pharmaceutical compositions, it is important that a reliable, reproducible and constant plasma concentration profile of the active pharmaceutical ingredient is provided following administration to a patient.

Chemical stability, solid state stability, and “shelf life” of the active pharmaceutical ingredient are important properties for a pharmaceutical active compound. The active pharmaceutical ingredient, and compositions containing it, should be capable of being effectively stored over appreciable periods of time, without exhibiting a significant change in the physico-chemical characteristics of the active pharmaceutical ingredient, e.g. its chemical composition, density, hygroscopicity and solubility. Thus, in the manufacture of commercially viable and pharmaceutically acceptable drug compositions, it is important, wherever possible, to provide the active pharmaceutical ingredient in a stable form.

New salt forms of a pharmaceutical agent can further the development of formulations for the treatment of illnesses. For instance, solid forms of salts of a compound are known in the pharmaceutical art to affect, for example, the solubility, dissolution rate, bioavailability, chemical and physical stability, flowability, fractability, and compressibility of the compound as well as the safety and efficacy of drug products based on the compound.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Solvent medium and mode of crystallization play very important role in obtaining a new salt or a crystalline form over the other.

The discovery of novel salts in solid state forms, including amorphous and crystalline forms, of pharmaceutically useful compound provides a new opportunity to improve the performance characteristics of a pharmaceutical product. It also adds value to the material that a formulation scientist can use the same for designing, for example, a pharmaceutical dosage form of a drug with a targeted release profile or other desired characteristic. Novel solid forms, including amorphous form and crystal forms, of eletriptan hemioxalate have now been discovered.

### SUMMARY OF THE INVENTION

The novel hemioxalate salt of (R)-5-[2-(phenylsulfonyl)ethyl]-3-[(1-methyl-2-pyrrolidinyl)methyl]-1H-indole, that is, eletriptan hemioxalate, is provided.

According to one aspect, the present invention provides novel solid state forms, including amorphous and crystalline forms, of eletriptan hemioxalate.

In another aspect, eletriptan hemioxalate in a solid state is provided. In another aspect, eletriptan hemioxalate in a crystalline form is provided. In yet another aspect, eletriptan hemioxalate in an amorphous form is provided. In another aspect, the solid state forms of eletriptan hemioxalate may exist in an anhydrous and/or solvent-free form or as a hydrate and/or a solvate form.

It has also been found that the novel eletriptan hemioxalate is useful intermediate in the preparation of eletriptan free base or a pharmaceutically acceptable salt thereof in high purity. The solid forms of eletriptan hemioxalate have good flow properties and are far more stable at room temperature, enhanced temperature and at relative high humidities and in aqueous media, and so, the novel solid forms of eletriptan hemioxalate are suitable for formulating eletriptan.

In another aspect, the present invention encompasses a process for preparing the novel eletriptan hemioxalate salt comprising contacting eletriptan free base with oxalic acid in a suitable solvent under suitable conditions, and isolating the eletriptan hemioxalate as solid.

The suitable solvent is selected from the group comprising water, alcohols, ketones, chlorinated hydrocarbons, hydrocarbons, nitriles, esters, ethers, polar aprotic solvents, and mixtures thereof. Preferable solvents are water, alcohols, ketones, polar aprotic solvents, and mixtures thereof, and most preferably acetone, methanol, ethanol, isopropanol, and mixtures thereof.

In another aspect, the present invention provides a method for the use of novel eletriptan hemioxalate salt for the treatment of patients suffering from depression, anxiety, eating disorders, obesity, drug abuse, cluster headache, migraine, chronic paroxysmal hemicrania and headache associated with vascular disorders, pain, and other disorders arising from deficient serotonergic neurotransmission; comprising administering the novel solid forms of eletriptan hemioxalate, or a pharmaceutical composition that comprises novel solid forms of eletriptan hemioxalate, along with pharmaceutically acceptable excipients.

In another aspect, the present invention provides pharmaceutical compositions comprising a therapeutically effective amount of the solid forms of eletriptan hemioxalate of the present invention, and one or more pharmaceutically acceptable excipients.

In another aspect, the present invention provides pharmaceutical compositions comprising the solid forms of eletriptan hemioxalate prepared according to processes of the present invention in any of its embodiments and one or more pharmaceutically acceptable excipients.

In yet another aspect, the present invention further encompasses a process for preparing a pharmaceutical formulation comprising combining any one of the solid forms of eletriptan hemioxalate prepared according to processes of the present invention in any of its embodiments, with one or more pharmaceutically acceptable excipients.

In another aspect, the substantially pure solid forms of eletriptan hemioxalate disclosed herein for use in the pharmaceutical compositions of the present invention, wherein 90 volume-percent of the particles (D₉₀) have a size of less than or equal to about 500 microns, specifically less than or equal to about 400 microns, specifically less than or equal to about 300 microns, more specifically less than or equal to about 200 microns, specifically less than or equal to about 150 microns, still more specifically less than or equal to about 100 microns, specifically less than equal to about 50 microns, and most specifically less than or equal to about 15 microns.

Unless otherwise indicated, the following definitions are set forth to illustrate and define the meaning and scope of the various terms used to describe the invention herein.

The term “solid form of eletriptan hemioxalate disclosed herein” includes crystalline forms, amorphous form, hydrated and solvated forms of eletriptan hemioxalate.

The term “crystalline polymorph” refers to a crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous form of eletriptan hemioxalate in accordance with the present invention preferably contains less than about 10% crystalline forms of eletriptan hemioxalate, more preferably less than 5% crystalline form of eletriptan hemioxalate, and still more preferably is essentially free of crystalline forms of eletriptan hemioxalate. “Essentially free of crystalline forms of eletriptan hemioxalate” means that no crystalline polymorph forms of eletriptan hemioxalate can be detected within the limits of a powder X-ray diffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The expression “pharmaceutically acceptable salt” is meant those salts which are, within the scope of sound medical judgment, suitable for use in contact with the tissues of humans and lower animals without undue toxicity, irritation, allergic response and the like, commensurate with a reasonable benefit/risk ratio, and effective for their intended use.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art. If required, other binders may also be included in the present invention.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, combinations thereof and other such materials known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, macrocrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

As used herein, D_{X} means that X percent of the particles have a diameter less than a specified diameter D. Thus, a D₉₀ of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

The term “water content” refers to the content of water based upon the Loss on Drying method as described in Pharmacopeial Forum, Vol. 24, No. 1, page 5438 (January-February 1998), the Karl Fisher assay for determining water content or thermogravimetric analysis (TGA). The calculation of water content is based upon the percent of weight that is lost by drying.

The term “Anti-solvent” refers to a solvent which when added to an existing solution of a substance reduces the solubility of the substance.

The term “alcohol solvents” include, but are not limited to, C₁ to C₈ straight or branched chain alcohol solvents such as methanol, ethanol, n-propanol, isopropanol, n-butanol, isobutanol, tert-butanol, amyl alcohol, hexanol, and mixtures thereof.

The term “ketone solvents” include, but are not limited to, acetone, methyl ethyl ketone, methyl isobutyl ketone, methyl tert-butyl ketone and the like, and mixtures thereof.

The term “nitrile solvents” include, but are not limited to, acetonitrile and the like, and mixtures thereof.

The term “ester solvents” include, but are not limited to, ethyl acetate, methyl acetate, isopropyl acetate, tert-butyl methyl acetate and ethyl formate, and the like and mixtures thereof.

The term “chlorinated hydrocarbon solvents” include, but are not limited to, methylene chloride, ethylene dichloride, chloroform, carbon tetrachloride, and mixtures thereof.

The term “ether solvents” include, but are not limited to, tetrahydrofuran, dioxane, diethyl ether, diisopropyl ether, monoglyme, diglyme and the like, and mixtures thereof.

The term “hydrocarbon solvents” means both aliphatic and aromatic hydrocarbon solvents include, but are not limited to, n-pentane, n-hexane, n-heptane and isomers thereof, cyclohexane, toluene and xylene and the like, and mixtures thereof.

The term “polar aprotic solvents” include, but are not limited to, N,N-dimethylformamide, N,N-dimethylacetamide, dimethylsulfoxide, and mixtures thereof.

By “substantially pure” is meant having purity greater than about 98%, specifically greater than about 99%, more specifically greater than about 99.5%, and still more specifically greater than about 99.9% measured by HPLC.

### BRIEF DESCRIPTION OF THE DRAWINGS

FIG. 1 is a characteristic powder X-ray diffraction (XRD) pattern of crystalline eletriptan hemioxalate.

FIG. 2 is a characteristic differential scanning calorimetric (DSC) thermogram of crystalline eletriptan hemioxalate.

FIG. 3 is a characteristic powder X-ray diffraction (XRD) pattern of amorphous eletriptan hemioxalate.

FIG. 4 is a characteristic infra red (IR) spectrum of eletriptan hemioxalate.

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-50° 2-theta; step width=0.01579°; and measuring time per step=0.11 sec.

The DSC thermogram of eletriptan salt was recorded on TA instrument, Q-1000 differential scanning colorimeter in a temperature range of 25-220° C. with a heating rate of 20° C./minute.

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 3800 to 650 cm⁻¹.

### DETAILED DESCRIPTION OF THE INVENTION

According to one aspect of the present invention, there is provided a novel hemioxalate salt of (R)-5-[2-(phenylsulfonyl)ethyl]-3-[(1-methyl-2-pyrrolidinyl)methyl]-1H-indole, that is, eletriptan hemioxalate, of formula I:

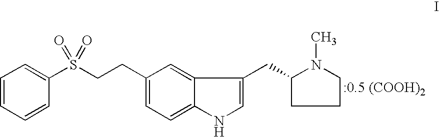

According to another aspect of the present invention, there is provided novel solid state forms, including amorphous and crystalline forms, of eletriptan hemioxalate.

In another aspect, eletriptan hemioxalate in a solid state is provided. In another aspect, eletriptan hemioxalate in a crystalline form is provided. In yet another aspect, eletriptan hemioxalate in an amorphous form is provided. In another aspect, the solid state forms of eletriptan hemioxalate may exist in an anhydrous and/or solvent-free form or as a hydrate and/or a solvate form. Such solvated or hydrated forms may be present as hemi-, mono-, sesqui-, di- or tri-solvates or hydrates. Solvates and hydrates may be formed as a result of solvents used during the formation of the eletriptan hemioxalate becoming imbedded in the solid lattice structure. Because formation of the solvates and hydrates occurs during the preparation of eletriptan hemioxalate, formation of a particular solvated or hydrated form depends greatly on the conditions and method used to prepare the salt. Solvents should be pharmaceutically acceptable.

In another embodiment, the novel eletriptan hemioxalate is useful intermediate in the preparation of eletriptan free base or a pharmaceutically acceptable salt thereof, preferably eletriptan hydrobromide, in high purity. The solid forms of eletriptan hemioxalate have good flow properties and are far more stable at room temperature, enhanced temperature and at relative high humidities and in aqueous media, and so, the novel solid forms of eletriptan hemioxalate are suitable for formulating eletriptan.

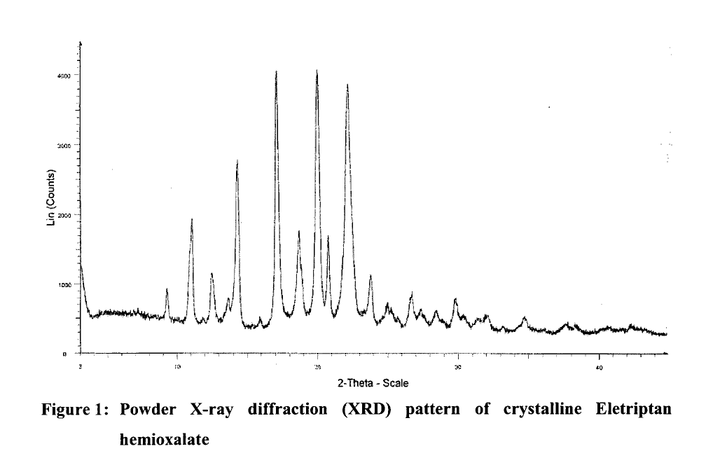

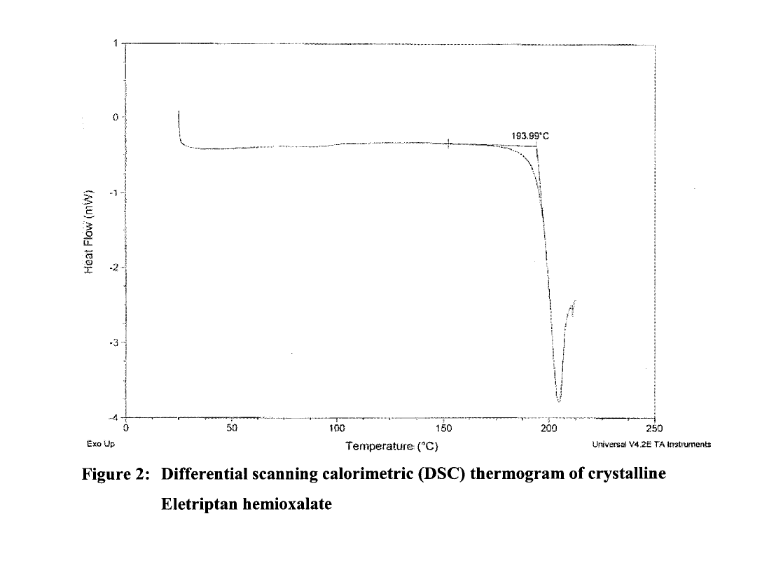

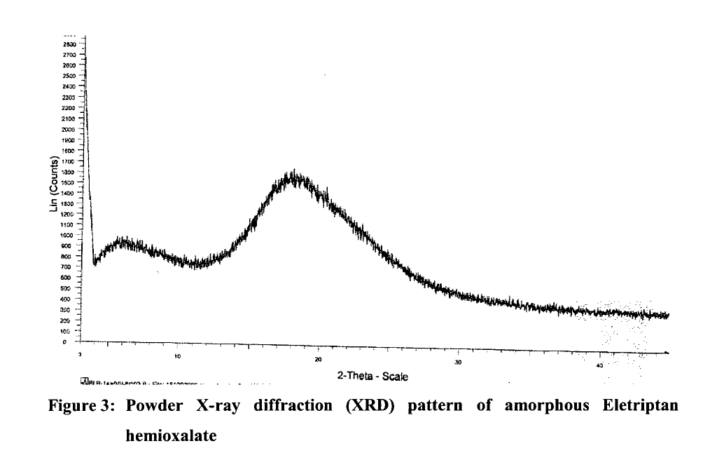

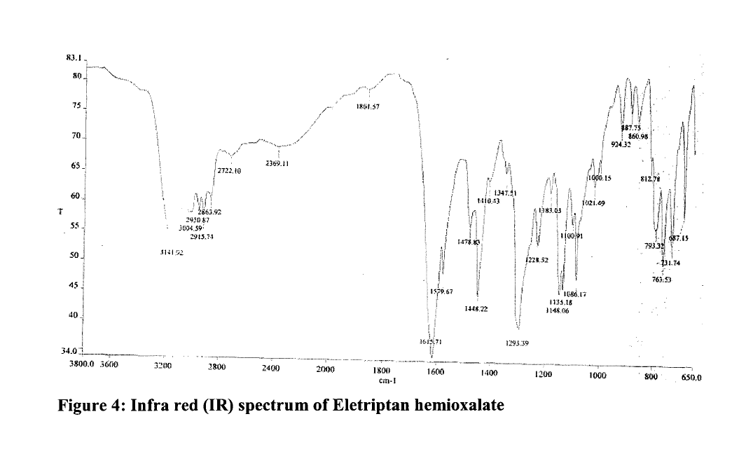

In an embodiment, the eletriptan hemioxalate salt is characterized by at least one, or more, of the following properties:
i) a powder X-ray diffraction pattern substantially in accordance with FIG. 1;
ii) a powder X-ray diffraction pattern having peaks at about 11.01, 14.29, 17.09 and 19.98±0.2 degrees 2-theta substantially as depicted in FIG. 1;
iii) a powder X-ray diffraction pattern having additional peaks at about 9.23, 12.46, 13.62, 18.64, 18.87, 20.76, 22.16, 22.50, 23.81, 26.69 and 29.83±0.2 degrees 2-theta substantially as depicted in FIG. 1;
iv) a differential scanning calorimetric (DSC) thermogram substantially in accordance with FIG. 2;
v) a powder X-ray diffraction pattern substantially in accordance with FIG. 3;
vi) an IR spectrum substantially in accordance with FIG. 4; and
vii) an IR spectrum having absorption bands at about 3141, 3004, 2950, 2915, 2863, 1615, 1579, 1478, 1448, 1293, 1228, 1183, 1148, 1135, 1086, 1100, 1021, 812, 793, 763, 731 and 687±1 cm⁻¹.

According to another aspect of the present invention, there is provided a process for the preparation of eletriptan hemioxalate; comprising the steps of:
a) contacting eletriptan free base with oxalic acid in a suitable solvent to produce a reaction mass containing eletriptan hemioxalate salt;
b) optionally, heating the reaction mass obtained in step-(a); and
c) substantially removing the solvent from the reaction mass obtained in step-(a) or step-(b) to afford solid form of eletriptan hemioxalate; and/or
d) isolating solid form of eletriptan hemioxalate salt by forcible or spontaneous crystallization;
e) optionally, heating the solid form of eletriptan hemioxalate obtained in step-(c) or step-(d) at a temperature of about 120° C. to about 210° C. until the solid form is converted to amorphous form.

The suitable solvent used in step-(a) is selected from the group comprising water, alcohols, ketones, chlorinated hydrocarbons, hydrocarbons, nitriles, esters, ethers, polar aprotic solvents, and mixtures thereof.

Specifically, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically the solvent is selected from the group consisting of water, acetone, methanol, ethanol, n-propanol, isopropanol, n-butanol, isobutanol, tert-butanol, amyl alcohol, N,N-dimethylformamide, N,N-dimethylacetamide, dimethylsulfoxide, and mixtures thereof; and most specifically the solvent is selected from the group consisting of water, acetone, methanol, ethanol, isopropanol, and mixtures thereof.

The reaction in step-(a) is carried out at a temperature of below about reflux temperature of the solvent used, preferably at about 0° C. to about 80° C., more preferably at about 20° C. to about 60° C.

The reaction mass in step-(b) is preferably heated at a temperature of about 40° C. to the reflux temperature of the solvent used for at least 20 minutes, and more preferably at the reflux temperature of the solvent used from about 30 minutes to about 5 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution or a combination thereof.

The term “Anti-solvent” refers to a solvent which when added to an existing solution of a substance reduces the solubility of the substance.

Exemplary anti-solvents include, but are not limited to, ethers, hydrocarbons and mixtures thereof. Exemplary ether solvents include, but are not limited to, diisopropyl ether, diethyl ether, tetrahydrofuran, dioxane, and the like, and mixtures thereof. Exemplary hydrocarbon solvents include, but are not limited to, n-pentane, n-hexane and n-heptane and their isomers, cyclohexane, toluene, xylene, and mixtures thereof. Preferable anti-solvents are ether solvents, and most preferably diisopropyl ether, diethyl ether and mixtures thereof.

Preferably, the crystallization is carried out by cooling the solution at a temperature of below about 30° C., more preferably at a temperature of about 0° C. to about 25° C., and most preferably at about 0° C. to about 10° C.

The pure solid form of eletriptan hemioxalate obtained in step-(d) may be recovered by conventional techniques known in the art such as filtration, filtration under vacuum, decantation, and centrifugation, or a combination thereof.

In one embodiment, the pure solid form of eletriptan hemioxalate can be isolated by filtration employing a filtration media of, for example, a silica gel or celite.

The pure solid form of eletriptan hemioxalate obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

According to another aspect of the present invention, there is provided a solid form of eletriptan hemioxalate salt characterized by at least one, or more, of the following properties:
i) a powder X-ray diffraction pattern substantially in accordance with FIG. 1;
ii) a powder X-ray diffraction pattern having peaks at about 11.01, 14.29, 17.09 and 19.98±0.2 degrees 2-theta substantially as depicted in FIG. 1;
iii) a powder X-ray diffraction pattern having additional peaks at about 9.23, 12.46, 13.62, 18.64, 18.87, 20.76, 22.16, 22.50, 23.81, 26.69 and 29.83+0.2 degrees 2-theta substantially as depicted in FIG. 1; and
iv) a differential scanning calorimetric (DSC) thermogram substantially in accordance with FIG. 2.

According to another aspect of the present invention, a process for the preparation of solid form of eletriptan hemioxalate salt is provided, comprising the steps of:
a) providing a solution of eletriptan free base in a suitable solvent or a mixture of suitable solvents;
b) combining the solution obtained in step-(a) with oxalic acid; and
c) optionally, heating the reaction mass obtained in step-(b);
d) isolating and/or recovering crystalline eletriptan hemioxalate salt.

The process can produce crystalline eletriptan hemioxalate salt in substantially pure form.

The term “substantially pure crystalline eletriptan hemioxalate salt” refers to the crystalline eletriptan hemioxalate salt having purity greater than about 98%, specifically greater than about 99%, more specifically greater than about 99.5% and still more specifically greater than about 99.9% (measured by HPLC).

In a preferred embodiment, the crystalline form of eletriptan hemioxalate obtained according the present invention having water content less than about 5% by weight, specifically less than about 3.5% by weight, and more specifically less than about 1% by weight, and still more specifically is essentially free from water.

The crystalline eletriptan hemioxalate salt is stable, consistently reproducible and has good flow properties, and which is particularly suitable for bulk preparation and handling, and so, the novel crystalline eletriptan hemioxalate salt is suitable for formulating eletriptan. Moreover, the crystalline eletriptan hemioxalate salt is useful intermediate in the preparation of eletriptan or a pharmaceutically acceptable salt thereof, preferably eletriptan hydrobromide, in high purity.

The suitable solvent used in step-(a) is selected from the group comprising water, alcohols, ketones, nitriles, esters, tetrahydrofuran, and mixtures thereof.

Specifically, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, tetrahydrofuran, and mixtures thereof; more specifically the solvent is selected from the group consisting of water, acetone, methanol, ethanol, n-propanol, isopropanol, n-butanol, isobutanol, tert-butanol, ethyl acetate, acetonitrile, and mixtures thereof; and most specifically the solvent is selected from the group consisting of water, acetone, methanol, ethanol, isopropanol, ethyl acetate, and mixtures thereof.

Step-(a) of providing a solution of eletriptan free base includes dissolving any form of eletriptan free base (solid or residue) in the suitable solvent, or obtaining an existing solution from a previous processing step.

Preferably the eletriptan free base is dissolved in the solvent at a temperature of about 0° C. to about the reflux temperature of the solvent used, more preferably at about 20° C. to about 100° C., and still more preferably at about 25° C. to about 80° C.

Alternatively, the solution in step-(a) may be prepared by treating an acid addition salt of eletriptan with a base to liberate eletriptan free base and dissolving the eletriptan free base in the suitable solvent.

The acid addition salts can be derived from a therapeutically acceptable acid such as hydrochloric acid, hydrobromic acid, sulfuric acid, nitric acid, phosphoric acid, acetic acid, propionic acid, succinic acid, maleic acid, fumaric acid, benzenesulfonic acid, toluenesulfonic acid, citric acid, glutaric acid, citraconic acid, glutaconic acid, and tartaric acid can be used.

The treatment of an acid addition salt with base is carried out in any solvent and the selection of solvent is not critical. A wide variety of solvents such as chlorinated solvents, hydrocarbon solvents, ether solvents, alcohol solvents, ketone solvents, ester solvents etc., can be used.

The base can be inorganic or organic. Preferable base is an inorganic base selected from ammonia, alkali metal hydroxides, carbonates and bicarbonates. Preferable alkali metal is sodium or potassium.

The oxalic acid in step-(b) may be used directly or in the form of oxalic acid dissolved in a suitable solvent. The suitable solvent used for dissolving oxalic acid is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, tetrahydrofuran, and mixtures thereof.

Usually, about 0.2 to 2.0 moles, specifically, about 0.4 to 1.2 moles of oxalic acid is used per 1 mole of eletriptan free base.

The combining of the solution with oxalic acid in step-(b) is done in a suitable order, for example, the solution is added to the oxalic acid, or alternatively, the oxalic acid is added to the solution. The addition is carried out drop wise, in one portion, or in more than one portion. In one embodiment, addition is carried out at a temperature of below about 60° C. for at least 15 minutes, and more specifically at a temperature of about 15° C. to about 35° C. from about 20 minutes to about 2 hours. After completion of the addition process, the resulting mass is stirred for at least 20 minutes, more specifically about 30 minutes to about 16 hours, at a temperature of about 20° C. to about 35° C.

The heating in step-(c) is carried out at a temperature of about 40° C. to the reflux temperature of the solvent used for at least 20 minutes, and more preferably at a temperature of about 40° C. to about 80° C. from about 30 minutes to about 4 hours.

The solution obtained in step-(b) or step-(c) can be optionally subjected to carbon treatment or silica gel treatment. The carbon treatment or silica gel treatment can be carried out by methods known in the art, for example by stirring the solution with finely powdered carbon or silica gel at a temperature of below about 70° C. for at least 15 minutes, preferably at a temperature of about 40° C. to about 70° C. for at least 30 minutes; and filtering the resulting mixture through hyflo to obtain a filtrate containing eletriptan hemioxalate by removing charcoal or silica gel. Preferably, finely powdered carbon is an active carbon.

The isolation of pure crystalline eletriptan hemioxalate salt in step-(d) may be carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

Exemplary anti-solvents include, but are not limited to, ethers, hydrocarbons and mixtures thereof. Exemplary ether solvents include, but are not limited to, diisopropyl ether, diethyl ether, tetrahydrofuran, dioxane, and the like, and mixtures thereof. Exemplary hydrocarbon solvents include, but are not limited to, n-pentane, n-hexane and n-heptane and their isomers, cyclohexane, toluene, xylene, and mixtures thereof. Preferable anti-solvents are ether solvents, and most preferably diisopropyl ether, diethyl ether and mixtures thereof.

In an embodiment, the crystallization is carried out by stirring the solution at a temperature of below 30° C. for at least 20 minutes, and more preferably at about 0° C. to about 30° C. from about 1 hour to about 10 hours.

The pure crystalline form of eletriptan hemioxalate salt obtained in step-(d) may be recovered by conventional techniques known in the art such as filtration, filtration under vacuum, decantation, and centrifugation, or a combination thereof.

In one embodiment, the pure crystalline form of eletriptan hemioxalate salt can be isolated by filtration employing a filtration media of, for example, a silica gel or celite.

The pure crystalline form of eletriptan hemioxalate salt obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The total purity of the crystalline form of eletriptan hemioxalate salt obtained by the process disclosed herein is of greater than about 99%, specifically greater than about 99.5%, and more specifically greater than about 99.95% as measured by HPLC.

According to another aspect of the present invention, there is provided an amorphous form of eletriptan hemioxalate salt.

Amorphous form of eletriptan hemioxalate salt is characterized by a powder XRD pattern substantially in accordance with FIG. 3. The X-ray powder diffraction pattern shows no peaks, thus demonstrating the amorphous nature of the product.

According to another aspect of the present invention, there is provided a process for preparing amorphous form of eletriptan hemioxalate comprising heating crystalline eletriptan hemioxalate until the crystalline form is converted to amorphous form.

The conversion to amorphous form occurs at a temperature of above about 120° C., preferably at about 130° C. to about 210° C., and more preferably at about 180° C. to about 200° C.

The heating is carried out for at least about 30 minutes, usually about 45 minutes to about 15 hours and typically about 1 hour to about 5 hours.

After completion of the heating process, the resulting material is preferably cooled at a temperature of below about 50° C. for at least 30 minutes, and more preferably at a temperature of about 20° C. to about 40° C. from about 1 hour to 5 hours.

Pharmaceutically acceptable salts of eletriptan can be prepared in high purity by using the solid form of substantially pure eletriptan hemioxalate obtained by the method disclosed herein, by known methods, for example as described in U.S. Pat. No. 5,545,644.

Preferable pharmaceutically acceptable salts of eletriptan include, but are not limited to, hydrochloride, hydrobromide, hemisulphate, phosphate, hemifumarate, fumarate, hemisuccinate, succinate, maleate, fumarate, besylate, tosylate, tartrate, and more preferably hydrobromide.

Eletriptan free base or an acid addition salt thereof used as starting material can be obtained by processes described in the prior art, for example by the process described in the U.S. Pat. No. 5,545,644.

Karl Fisher analysis, which is well known in the art, is also used to determine the quantity of water in a sample.

According to another aspect of the present invention, there is provided a process for the preparation of substantially pure eletriptan hydrobromide, comprising the steps of:
a) providing a solution or suspension of eletriptan hemioxalate in a suitable solvent or a mixture of suitable solvents;
b) combining the solution or suspension obtained in step-(a) with hydrobromic acid; and
c) optionally, heating the reaction mass obtained in step-(b); and
d) isolating and/or recovering substantially pure eletriptan hydrobromide from the reaction mass.

The term “substantially pure eletriptan hydrobromide” refers to the eletriptan hydrobromide having purity greater than about 99%, specifically greater than about 99.5%, more specifically greater than about 99.9% and still more specifically greater than about 99.95% (measured by HPLC).

The suitable solvent used in step-(a) is selected from the group comprising water, alcohols, ketones, esters, and mixtures thereof.

Specifically, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof; more specifically, the solvent is selected from the group consisting of water, acetone, methanol, ethanol, n-propanol, isopropanol, n-butanol, isobutanol, tert-butanol, amyl alcohol, acetone, ethyl acetate, and mixtures thereof; and most specifically, the solvent is selected from the group consisting of water, acetone, methanol, ethanol, isopropanol, ethyl acetate, and mixtures thereof.

Step-(a) of providing a solution of eletriptan hemioxalate includes dissolving any form of eletriptan hemioxalate in the suitable solvent, or obtaining an existing solution from a previous processing step. The eletriptan hemioxalate is preferably used in the form a solid (crystalline or amorphous), and more preferably in the form a crystalline solid.

Preferably the eletriptan hemioxalate is dissolved in the solvent at a temperature of about 0° C. to about the reflux temperature of the solvent used, more preferably at about 20° C. to about 100° C., and still more preferably at about 25° C. to about 80° C.

Step-(a) of providing a suspension of eletriptan hemioxalate includes suspending any form of eletriptan hemioxalate in the solvent under stirring. Preferably the suspension is stirred for at least 30 minutes at below boiling temperature of the solvent used, and more preferably for 1 hour to 10 hours at about 25° C. to about 80° C.

In an embodiment, the hydrobromic acid used in step-(b) may be in the form of concentrated hydrobromic acid or aqueous hydrobromic acid or in the form of hydrobromic acid diluted in a suitable solvent. The solvent used for diluting hydrobromic acid is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof.

Usually, about 0.8 to 1.2 moles, specifically, about 0.9 to 1.0 moles of hydrobromic acid is used per 1 mole of eletriptan hemioxalate.

The combining of the solution or suspension with hydrobromic acid in step-(b) is done in a suitable order, for example, the solution or suspension is added to the hydrobromic acid, or alternatively, the hydrobromic acid is added to the solution or suspension. The addition is carried out drop wise, in one portion, or in more than one portion. In one embodiment, addition is carried out at a temperature of below about 60° C. for at least 15 minutes, and more specifically at a temperature of about 15° C. to about 35° C. from about 20 minutes to about 2 hours. After completion of the addition process, the resulting mass is stirred for at least 20 minutes, more specifically about 30 minutes to about 10 hours, at a temperature of about 20° C. to about 35° C.

The heating in step-(c) is carried out at a temperature of about 40° C. to the reflux temperature of the solvent used for at least 20 minutes, and more preferably at a temperature of about 40° C. to about 80° C. from about 30 minutes to about 4 hours.

The solution obtained in step-(b) or step-(c) can be optionally subjected to carbon treatment or silica gel treatment. The carbon treatment or silica gel treatment can be carried out by methods known in the art, for example by stirring the solution with finely powdered carbon or silica gel at a temperature of below about 70° C. for at least 15 minutes, preferably at a temperature of about 40° C. to about 70° C. for at least 30 minutes; and filtering the resulting mixture through hyflo to obtain a filtrate containing eletriptan hydrobromide by removing charcoal or silica gel. Preferably, finely powdered carbon is an active carbon.

The isolation of substantially pure eletriptan hydrobromide in step-(d) may be carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

In an embodiment, the crystallization is carried out by stirring the solution at a temperature of below 35° C. for at least 20 minutes, and more preferably at about 0° C. to about 30° C. from about 1 hour to about 10 hours.

The substantially pure eletriptan hydrobromide obtained in step-(d) may be recovered by conventional techniques known in the art such as filtration, filtration under vacuum, decantation, and centrifugation, or a combination thereof.

In one embodiment, the pure eletriptan hydrobromide can be isolated by filtration employing a filtration media of, for example, a silica gel or celite.

The pure eletriptan hydrobromide obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The total purity of the substantially pure eletriptan hydrobromide obtained by the process disclosed herein is of greater than about 99%, specifically greater than about 99.5%, and more specifically greater than about 99.95% as measured by HPLC.

In one embodiment, the substantially pure solid state forms of eletriptan hemioxalate disclosed herein for use in the pharmaceutical compositions of the present invention, wherein 90 volume-percent of the particles (D₉₀) have a size of less than or equal to about 500 microns, specifically less than or equal to about 400 microns, specifically less than or equal to about 300 microns, more specifically less than or equal to about 200 microns, specifically less than or equal to about 150 microns, still more specifically less than or equal to about 100 microns, specifically less than or equal to about 50 microns and most specifically less than or equal to about 15 microns.

In another embodiment, the particle sizes of substantially pure solid state forms of eletriptan hemioxalate can be achieved via a mechanical process of reducing the size of particles which includes any one or more of cutting, chipping, crushing, milling, grinding, micronizing, trituration or other particle size reduction methods known in the art, to bring the solid state forms the desired particle size range.

According to another aspect of the present invention, there is provided a method for the use of novel eletriptan hemioxalate for the treatment of patients suffering from depression, anxiety, eating disorders, obesity, drug abuse, cluster headache, migraine, chronic paroxysmal hemicrania and headache associated with vascular disorders, pain, and other disorders arising from deficient serotonergic neurotransmission; comprising administering a therapeutically effective amount of the novel solid forms of eletriptan hemioxalate, or a pharmaceutical composition that comprises a therapeutically effective amount of the novel solid forms of eletriptan hemioxalate, along with pharmaceutically acceptable excipients.

According to another aspect of the present invention, there is provided pharmaceutical compositions comprising the eletriptan hemioxalate salt and one or more pharmaceutically acceptable excipients.

According to another aspect of the present invention, there is provided pharmaceutical compositions comprising the eletriptan hemioxalate salt prepared according to processes of the present invention in any of its embodiments and one or more pharmaceutically acceptable excipients.

According to another aspect of the present invention, there is provided a process for preparing a pharmaceutical formulation comprising combining any one of the solid state forms of eletriptan hemioxalate prepared according to processes of the present invention in any of its embodiments, with one or more pharmaceutically acceptable excipients.

Yet another embodiment of the present invention is directed to pharmaceutical compositions comprising at least a therapeutically effective amount of any one of the substantially pure solid state forms of eletriptan hemioxalate of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The solid state forms of eletriptan hemioxalate of the present invention may also be administered as suppositories, ophthalmic ointments and suspensions, and parenteral suspensions, which are administered by other routes. The dosage forms may contain any one of the solid state forms of eletriptan hemioxalate of the present invention as is or, alternatively, may contain any one of the solid state forms of eletriptan hemioxalate of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In another embodiment of the present invention, there is provided pharmaceutical compositions comprising crystalline eletriptan hemioxalate and one or more pharmaceutically acceptable excipients.

In another embodiment of the present invention, there is provided pharmaceutical compositions comprising amorphous eletriptan hemioxalate and one or more pharmaceutically acceptable excipients.

Capsule dosages will contain the solid state forms of eletriptan hemioxalate of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The following examples are given for the purpose of illustrating the present invention and should not be considered as limitation on the scope or spirit of the invention.

### EXAMPLES

### Example 1

### Preparation of Crystalline Eletriptan Hemioxalate

Eletriptan base (18.36 g, 0.048 moles) was taken in acetone (55 ml) at 25-30° C. followed by the drop wise addition of a solution of oxalic acid (3 g, 0.023 moles) dissolved in acetone (18 ml) at 25-30° C. in 30-60 minutes. The pH of resulted solution was adjusted about 6-7 by adding solution of oxalic acid in acetone. The reaction mixture was stirred for 60 minutes at 25-30° C. The resulted solid was filtered and washed with acetone (18 ml) and further dried in air oven at 35-40° C. to give 15 g of eletriptan hemioxalate as crystalline solid (HPLC Purity: 98.8%).

### Example 2

### Preparation of Crystalline Eletriptan Hemioxalate

Eletriptan base (30 g, 0.078 moles) was taken in methanol (45 ml) at 25-30° C. followed by the drop wise addition of a solution of oxalic acid (4.9 g, 0.039 moles) dissolved in methanol (15 ml) at 25-30° C. in 30-60 minutes. The reaction mixture was stirred for 2 hours at 25-30° C. The resulted solid was filtered and washed with methanol (15 ml) and further dried in air oven at 35-40° C. to give 26 g of eletriptan hemioxalate as crystalline solid (HPLC Purity: 99.86%).

### Example 3

### Preparation of Amorphous Eletriptan Hemioxalate

Eletriptan hemioxalate (2 g, obtained in example 1) was heated at 195-200° C. under nitrogen atmosphere to melt. The molten eletriptan hemioxalate was further allowed to cool after 15 minutes at 25-30° C. for 1 hour and the sample was grinded to give amorphous eletriptan hemioxalate quantitatively.

### Example 4

### Preparation of Eletriptan Hydrobromide

Eletriptan hemioxalate (20.0 g, 0.044 moles) was taken in isopropanol (100 ml) at 25-30° C. This was followed by the drop wise addition of a solution of 48% aqueous hydrobromic acid (9.3 g, 0.037 moles) diluted in isopropanol (50 ml) at 25-30° C. in 30-60 minutes. The pH of resulted solution was adjusted to 6-7 by adding a solution of aqueous hydrobromic acid in isopropyl alcohol. The reaction mixture was stirred for 2 hours at 25-30° C. The resulted solid was filtered and washed with isopropanol (50 ml) and further dried under vacuum at 50-55° C. to give 20 g of eletriptan hydrobromide (HPLC Purity: 99.82%).

### Example 5

### Preparation of Eletriptan Hydrobromide

Eletriptan hemioxalate (2 g, 0.0046 moles) was taken in a mixture of acetone (2.5 ml) and water (10 ml) at 25-30° C. This was followed by the drop wise addition of a solution of 48% aqueous hydrobromic acid (0.93 g, 0.0037 moles) diluted in water (2 ml)) at 25-30° C. in 30 minutes. The pH of resulted solution was adjusted to 6-7 by adding solution of aqueous hydrobromic acid in water. The reaction mixture was stirred for 2 hours at 25-30° C. The resulted solid was filtered and washed with water (20 ml) and further dried under vacuum at 50-55° C. to give 1.75 g of eletriptan hydrobromide (HPLC Purity: 99.5%).

### Example 6

### Preparation of Eletriptan Hydrobromide

Eletriptan hemioxalate (10.0 g, 0.0.234 moles) was taken in water (50 ml) at 25-30° C. This was followed by the drop wise addition of a solution of 48% aqueous hydrobromic acid solution (3.85 g, 0.019 moles) diluted in water (10 ml)) at 25-30° C. in 30 minutes. The pH of resulted solution was adjusted to 6-7 by adding solution of aqueous hydrobromic acid in water. The reaction mixture was stirred for 2 hours at 25-30° C. The resulted solid was filtered and washed with water (40 ml) and further dried under vacuum at 50-55° C. to give 9.25 g of eletriptan hydrobromide (HPLC Purity: 99.6%).

### Example 7

### Preparation of Eletriptan Hydrobromide

Eletriptan hemioxalate (20.0 g, 0.044 moles) was taken in ethyl acetate (100 ml) at 25° C.-30° C. This was followed by the drop wise addition of 48% aqueous hydrobromic acid (9.3 g, 0.037 moles) at 25-30° C. in 30-60 minutes. The reaction mixture was stirred for 2 hours at 25-30° C. The resulted solid was filtered and washed with ethyl acetate (20 ml) and further dried under vacuum at 50-55° C. to give 19 g of eletriptan hydrobromide

1. A hemioxalate salt of (R)-5-[2-(phenylsulfonyl)ethyl]-3-[(1-methyl-2-pyrrolidinyl)methyl]-1H-indole, eletriptan hemioxalate, of formula I:

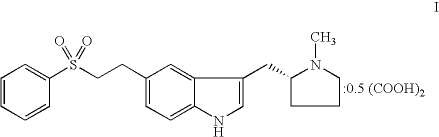

2. The eletriptan hemioxalate salt of claim 1, which is in a solid state form, wherein the solid state form is a crystalline form, an amorphous form, an anhydrous and/or solvent-free form, or a hydrate and/or a solvate form.

3. (canceled)

4. (canceled)

5. The eletriptan hemioxalate salt of claim 1, characterized by at least one, or more, of the following properties:
i) a powder X-ray diffraction pattern substantially in accordance with FIG. 1;
ii) a powder X ray diffraction pattern having peaks at above 11.01, 14.29, 17.09 and 19.98±0.2 degrees 2 theta;
iii) a powder X-ray diffraction pattern having additional peaks at about 9.23, 12.46, 13.62, 18.64, 18.87, 20.76, 22.16, 22.50, 23.81, 26.69 and 29.83±0.2 degrees 2-theta;
iv) a differential scanning calorimetric (DSC) thermogram substantially in accordance with FIG. 2;
v) a powder X-ray diffraction pattern substantially in accordance with FIG. 3;
vi) an IR spectrum substantially in accordance with FIG. 4; and
vii) an IR spectrum having absorption bands at about 3141, 3004, 2950, 2915, 2863, 1615, 1579, 1478, 1448, 1293, 1228, 1183, 1148, 1135, 1086, 1100, 1021, 812, 793, 763, 731 and 687±1 cm⁻¹.

6. A process for the preparation of eletriptan hemioxalate of claim 1, comprising the steps of:
a) contacting eletriptan free base with oxalic acid in a solvent to produce a reaction mass containing eletriptan hemioxalate salt, wherein the solvent is selected from the group comprising water, alcohols, ketones, chlorinated hydrocarbons, hydrocarbons, nitriles, esters, ethers, polar aprotic solvents, and mixtures thereof;
b) optionally, heating the reaction mass obtained in step-(a); and
c) substantially removing the solvent from the reaction mass obtained in step-(a) or step-(b) to produce a solid form of eletriptan hemioxalate; or
d) isolating the solid form of eletriptan hemioxalate salt from the reaction mass obtained in step-(a) or step-(b) by forcible or spontaneous crystallization;
e) optionally, heating the solid form of eletriptan hemioxalate obtained in step-(c) or step-(d) at a temperature of about 120° C. to about 210° C. until the solid form is converted to amorphous form.

7. (canceled)

8. The process of claim 6, wherein the solvent used in step-(a) is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, methylene chloride, ethylene dichloride, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; wherein the reaction in step-(a) is carried out at a temperature of below about reflux temperature of the solvent used wherein the reaction mass in step-(b) is heated at a temperature of about 40° C. to the reflux temperature of the solvent used for at least 20 minutes; wherein the removal of solvent in step-(c) is accomplished by evaporation, vacuum drying, spray drying, freeze drying or a combination thereof; wherein the crystallization in step-(d) is initiated by cooling, seeding, partial removal of the solvent from the solution, by adding an anti-solvent to the solution or a combination thereof; and wherein the solid form of eletriptan hemioxalate salt obtained in step-(d) is recovered by filtration, filtration under vacuum, decantation, and centrifugation, or a combination thereof.

9. (canceled)

10. (canceled)

11. (canceled)

12. (canceled)

13. (canceled)

14. (canceled)

15. (canceled)

16. (canceled)

17. (canceled)

18. A crystalline form of eletriptan hemioxalate salt, characterized by, one or more, of the following properties:
i) a powder X-ray diffraction pattern substantially in accordance with FIG. 1;
ii) a powder X-ray diffraction pattern having peaks at about 11.01, 14.29, 17.09 and 19.98±0.2 degrees 2-theta;
iii) a powder X-ray diffraction pattern having additional peaks at about 9.23, 12.46, 13.62, 18.64, 18.87, 20.76, 22.16, 22.50, 23.81, 26.69 and 29.83±0.2 degrees 2-theta; and
iv) a differential scanning calorimetric (DSC) thermogram substantially in accordance with FIG. 2.

19. A process for the preparation of crystalline form of eletriptan hemioxalate salt of claim 18, comprising:
a) providing a solution of eletriptan free base in a solvent or a mixture of solvents; wherein the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, tetrahydrofuran, and mixtures thereof;
b) combining the solution obtained in step-(a) with oxalic acid to produce a reaction mass containing eletriptan hemioxalate salt; and
c) optionally, heating the reaction mass obtained in step-(b);
d) isolating and/or recovering crystalline eletriptan hemioxalate salt from the reaction mass obtained in step-(b) or step-(c).

20. (canceled)

21. (canceled)

22. The process of claim 19, wherein the solvent used in step-(a) is selected from the group consisting of water, acetone, methanol, ethanol, isopropanol, ethyl acetate, and mixtures thereof; wherein the oxalic acid in step-(b) is used directly or in the form of oxalic acid dissolved in a suitable solvent selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, tetrahydrofuran, and mixtures thereof; wherein the combining in step-(b) is accomplished by adding the oxalic acid to the solution of eletriptan free base or by adding the eletriptan free base solution to the oxalic acid at a temperature of below about 60° C.; wherein the reaction mass obtained after addition of oxalic acid in step-(b) is further stirred for at least 20 minutes at a temperature of about 20° C. to about 35° C.; wherein the oxalic acid in step-(b) is used in a molar ratio of about 0.2 to 2.0 moles per 1 mole of eletriptan free base; wherein the heating in step-(c) is carried out at a temperature of about 40° C. to the reflux temperature of the solvent used for at least 20 minutes; wherein the isolation of pure crystalline eletriptan hemioxalate salt in step-(d) is initiated by cooling, seeding, partial removal of the solvent from the solution, by adding an anti-solvent to the solution, or a combination thereof; wherein the recovering in step-(d) is carried out by filtration, filtration under vacuum, decantation, and centrifugation, or a combination thereof; and wherein the crystalline form of eletriptan hemioxalate salt obtained has total purity of greater than about 99% as measured by HPLC.

23. (canceled)

24. (canceled)

25. (canceled)

26. (canceled)

27. The process of claim 19, wherein the addition in step-(b) is carried out at a temperature of about 15° C. to about 35° C. for about 20 minutes to about 2 hours; wherein the oxalic acid is used in a molar ratio of about 0.4 to 1.2 moles per 1 mole of eletriptan free base; wherein the isolation is carried out by stirring the solution at a temperature of below 30° C. for at least 20 minutes; and wherein the crystalline form of eletriptan hemioxalate salt obtained has total purity of about 99% to about 99.95%.

28. (canceled)

29. (canceled)

30. (canceled)

31. (canceled)

32. (canceled)

33. (canceled)

34. (canceled)

35. (canceled)

36. (canceled)

37. (canceled)

38. Amorphous form of eletriptan hemioxalate salt characterized by a powder XRD pattern substantially in accordance with FIG. 3.

39. A process for preparing amorphous eletriptan hemioxalate of claim 38, comprising heating a crystalline form of eletriptan hemioxalate at a temperature of above about 120° C. until the crystalline form is converted to amorphous form, and wherein the material obtained after heating process is cooled at a temperature of below about 50° C. for at least 30 minutes.

40. (canceled)

41. The process of claim 39, wherein the crystalline eletriptan hemioxalate is heated at a temperature of about 130° C. to about 210° C. for about 45 minutes to about 15 hours; and wherein the material obtained after heating process is cooled at a temperature of about 20° C. to about 40° C. for about 1 hour to 5 hours.

42. (canceled)

43. (canceled)

44. (canceled)

45. (canceled)

46. (canceled)

47. A process for the preparation of substantially pure eletriptan hydrobromide using eletriptan hemioxalate salt, comprising:
a) providing a solution or suspension of eletriptan hemioxalate in a solvent selected from the group comprising water, alcohols, ketones, esters, and mixtures thereof;
b) combining the solution or suspension obtained in step-(a) with hydrobromic acid to produce a reaction mass; and
c) optionally, heating the reaction mass obtained in step-(b); and
d) isolating and/or recovering substantially pure eletriptan hydrobromide from the reaction mass.

48. (canceled)

49. The process of claim 47, wherein the solvent used in step-(a) is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof; wherein the hydrobromic acid in step-(b) is used in the form of concentrated hydrobromic acid or aqueous hydrobromic acid or in the form of hydrobromic acid dissolved in a solvent; wherein the combining in step-(b) is accomplished by adding the hydrobromic acid to the solution or suspension of eletriptan hemioxalate, or by adding the solution or suspension of eletriptan hemioxalate to the hydrobromic acid, at a temperature of below about 60° C.; wherein the reaction mass obtained after addition of hydrobromic acid in step-(b) is further stirred for at least 20 minutes at a temperature of about 20° C. to about 35° C.; wherein the hydrobromic acid in step-(b) is used in a molar ratio of about 0.8 to 1.2 moles per 1 mole of eletriptan hemioxalate; wherein the heating in step-(c) is carried out at a temperature of about 40° C. to the reflux temperature of the solvent used for at least 20 minutes; wherein the isolation of pure eletriptan hydrobromide in step-(d) is initiated by cooling, seeding, partial removal of the solvent from the solution, by adding an anti-solvent to the solution, or a combination thereof; wherein the recovery in step-(d) is carried out by filtration, filtration under vacuum, decantation, and centrifugation, or a combination thereof; and wherein the eletriptan hydrobromide obtained has total purity of greater than about 99% measured by HPLC.

50. The process of claim 49, wherein the solvent used in step-(a) is selected from the group consisting of water, acetone, methanol, ethanol, isopropanol, ethyl acetate, and mixtures thereof; wherein the solvent used for diluting hydrobromic acid is selected from the group consisting of water methanol ethanol n-propanol, isopropanol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof; wherein the addition in step-(b) is carried out at a temperature of about 15° C. to about 35° C. for about 20 minutes to about 2 hours; wherein the hydrobromic acid is used in a molar ratio of about 0.9 to 1.0 moles per 1 mole of eletriptan hemioxalate; wherein the isolation in step-(d) is carried out by stirring the solution at a temperature of below 35° C. for at least 20 minutes; and wherein the eletriptan hydrobromide has total purity of about 99% to about 99.95%.

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A pharmaceutical composition comprising eletriptan hemioxalate salt or a solid state form thereof of claim 1, and one or more pharmaceutically acceptable excipients, wherein the pharmaceutical composition is a solid dosage form, an oral suspension, a liquid, a powder, an elixir or an injectable solution.

67. (canceled)

68. (canceled)

69. (canceled)

70. (canceled)

71. (canceled)

72. (canceled)

73. The pharmaceutical composition of claim 66, wherein the eletriptan hemioxalate salt has a D₉₀ particle size of less than or equal to about 500 microns.

74. The pharmaceutical composition of claim 73, wherein the D₉₀ particle size is less than or equal to about 400 microns, less than or equal to about 300 microns, less than or equal to about 200 microns, less than or equal to about 150 microns, less than or equal to about 100 microns, less than or equal to about 50 microns, or less than or equal to about 15 microns.

75. A method of treating a patient suffering from depression, anxiety, eating disorders, obesity, drug abuse, cluster headache, migraine, chronic paroxysmal hemicrania and headache associated with vascular disorders, pain, and other disorders arising from deficient serotonergic neurotransmission; comprising administering a therapeutically effective amount of the eletriptan hemioxalate salt of any one of claim 1 or a pharmaceutical composition that comprises a therapeutically effective amount of the eletriptan hemioxalate salt, along with pharmaceutically acceptable excipients.
